(19) United States
(12) Patent Application Publication
(10) Pub. No.: US 2017/0311900 A1
(43) Pub. Date: Nov. 2, 2017

(54) METHOD AND APPARATUS FOR USE IN MONITORING A PHYSIOLOGICAL CHARACTERISTIC OF A SUBJECT

(71) Applicant: KONINKLIJKE PHILIPS N.V., EINDHOVEN (NL)

(72) Inventors: Jarno Mikael Riistama, Eindhoven (NL); Gerhard Spekowius, Eindhoven (NL); Joseph Ernest Rock, Littleton, MA (US); Emile Josephus Carlos Kelkboom, Eindhoven (NL); Silviu Dovancescu, Eindhoven (NL)

(21) Appl. No.: 15/526,411

(22) PCT Filed: Nov. 4, 2015
(86) PCT No.: PCT/IB2015/058523

§ 371 Date: May 12, 2017

Related U.S. Application Data
(60) Provisional application No. 62/079,029, filed on Nov. 13, 2014.

(51) Int. Cl. A61B 5/00; A61B 5/08; A61B 5/053; A61B 5/02

(52) U.S. Cl. CPC A61B 5/7275; A61B 5/6805; A61B 5/02028; A61B 5/0537; A61B 5/0809

(57) ABSTRACT

There is provided a method for use in monitoring a physiological characteristic of a subject, the method comprising: obtaining a general variability measure of the physiological characteristic, wherein the general variability measure is based on a historical data set of values of the physiological characteristic from a plurality of further subjects; calculating a personalization factor specific to the subject, based on physiological data relating to the subject; generating at least one personalized abnormality criterion for the physiological characteristic, based on the obtained general variability measure and the calculated personalization factor; receiving a measured value of the physiological characteristic of the subject; and determining whether the received measured value is abnormal by comparing it to the at least one personalized abnormality criterion, wherein the received measured value is determined to be abnormal if it meets the at least one personalized abnormality criterion.

### TECHNICAL FIELD OF THE INVENTION

The invention relates to a method and apparatus for use in monitoring and identifying abnormal values of a physiological characteristic of a subject.

### BACKGROUND TO THE INVENTION

Pulmonary congestion is a clinical condition that is caused by a number of different diseases such as heart failure or kidney disease. It consists of an accumulation of fluid in the interstitial and alveolar space of the lung following increased blood pressure in the pulmonary capillary vessels that leads to leakage of water from the blood to the lung space. It causes fluid retention and fluid redistribution in the body and leads to symptoms like dyspnea, fatigue, and activity intolerance. Pulmonary congestion resulting from elevated left atrial and left ventricular filling pressures is a main reason for heart failure hospitalization. This condition has a progressive nature and clinical signs and symptoms of pulmonary oedema occur late, typically when the lung fluid has increased at least six-fold from the initial stage of interstitial oedema. This means that pulmonary oedema is often not detected early, and necessary treatment for the patient is delayed.

Bio-impedance measurements, obtained by a bio-impedance monitor using, for example, external electrodes or an implanted device to measure the resistance of biological tissue to a small alternating current flowing across a region of interest, e.g. the thorax, can be used to detect pulmonary congestion. The principle underlying this technique is the fact that the electrical impedance (resistance and reactance) of biological tissue is directly linked to the hydration and water content of the tissue, namely intra-cellular and extra-cellular water. When thoracic fluid accumulates (e.g. during pulmonary congestion), there will be a better conductance of the current resulting in a decreased impedance. By measuring the impedance at different frequencies the resistance of the extracellular water (Rₑ) can be estimated according to the Cole-Cole model. Therefore, measurements of the electrical properties of the tissue can indicate the amount of fluid present in that part of the body.

If external electrodes are used, impedance measurements are influenced by several factors including sensor placement, skin moisture, body dimensions and body posture. It has been found that body dimensions and fat mass are particularly relevant to thoracic impedance measurements, making such measurements subject-specific. Implantable devices are not affected by variations in electrode placement or skin moisture; however, it is known that the measurements made by such devices have variability due to less controlled measurement conditions (the patient may be unaware that a measurement is being taken and so measurements may be obtained in a variety of situations which would cause differing fluid distribution in the body, such as lying down, sitting, walking, or exercising). These factors, combined with the normal variability of bio-impedance measurements, make it challenging to determine when an impedance measurement for a specific patient is abnormal and thus indicative of excess fluid accumulation.

“Intrathoracic Impedance Monitoring in Patients with Heart Failure” by Yu et. al., Circulation 112 (2005), 841-848 describes an algorithm that can be used to detect abnormal measurements. The algorithm estimates the baseline impedance (BL) and the short term impedance (STA) on the basis of daily bio-impedance measurements. The baseline acts as a reference that is not sensitive to large deviations of the new measurements. On the other hand, the STA is a filtered estimate of the recent measurements and is more sensitive to large deviations of the measurement. The algorithm raises a flag if the cumulative sum of the difference BL−STA exceeds a certain threshold for consecutive measurements, where BL−STA>X. The disadvantage of the described algorithm is that it does not consider the subject-specific variability of the impedance measurements, e.g. X is constant for each patient. As noted above, it is known that the measurement variability is patient specific, which implies that this method is not optimal and prone to false alarms.

WO 2014/091426 describes an alternative algorithm for detecting abnormal measurements which defines and continuously updates a subject-specific range of normal variability for bio-impedance, using the measurements obtained from a subject. However; this algorithm requires measurement data to be gathered from a given subject for an amount of time before it is able to determine a personalized variability measure for that subject. Consequently, the increased accuracy resulting from the personalization cannot be immediately provided.

A reliable means of fluid content monitoring would be a valuable tool to improve outcomes in heart failure hospitalizations and reduce healthcare costs. There is therefore a need for an improved method and apparatus that can immediately provide a reliable determination of whether a bio-impedance measurement obtained for a given subject is abnormal for that subject, and thus indicative of ill-health (for example indicative of excessive fluid accumulation in a part of the body where the resistance is relatively low, or, on the other hand, indicative of dehydration of the subject where the resistance is relatively high). Such a method and apparatus could be used in a home or hospital-based monitoring system to detect the presence and progression of pulmonary congestion, as well as for monitoring improvements in the patient's condition as a result of receiving treatment. Such a method and apparatus could also be used in monitoring other physiological characteristics of a subject, such as weight, heart rate, blood pressure, temperature, etc., where low or alternatively high values of the physiological characteristic indicates that the subject has (or the degree to which the subject has) a physiological condition.

### SUMMARY OF THE INVENTION

According to a first aspect of the invention, there is provided a method for use in monitoring a physiological characteristic of a subject, the method comprising:

obtaining a general variability measure of the physiological characteristic, wherein the general variability measure is based on a historical data set of values of the physiological characteristic from a plurality of further subjects;

calculating a personalization factor specific to the subject, based on physiological data relating to the subject;

generating at least one personalized abnormality criterion for the physiological characteristic, based on the obtained general variability measure and the calculated personalization factor;

receiving a measured value of the physiological characteristic of the subject; and

determining whether the received measured value is abnormal by comparing it to the at least one personalized abnormality criterion, wherein the received measured value is determined to be abnormal if it meets the at least one personalized abnormality criterion.

Advantageously, the physiological data may be of a type which is already available for the subject and/or can be obtained immediately, such that abnormality determination criteria can be personalized to the subject requiring a sustained period of monitoring. This in turn increases the accuracy of the detection of abnormal values of the physiological characteristic.

In some embodiments the at least one personalized abnormality criterion comprises one or more of: a personalized maximum value of the physiological characteristic, such that a received measured value which is above the maximum value is determined to be abnormal; and a personalized minimum value of the physiological characteristic, such that a received measured value which is below the minimum value is determined to be abnormal.

In some embodiments the at least one personalized abnormality criterion comprises a personalized range of normal values of the physiological characteristic, such that a received measured value which is outside the range of normal values is determined to be abnormal. In some such embodiments the personalized range of normal values is defined by a personalized maximum value of the physiological characteristic and/or a personalized minimum value of the physiological characteristic.

In some embodiments generating the at least one personalized abnormality criterion comprises determining a baseline value of the physiological characteristic, and the at least one personalized abnormality criterion is generated based on the determined base line value, the obtained general variability measure, and the calculated personalization factor. In some such embodiments the baseline value is a personalized baseline value, which is determined using data specific to the subject.

In some embodiments generating the at least one personalized abnormality criterion comprises: defining at least one general abnormality criterion based on the obtained general variability measure; and adjusting the at least one general abnormality criterion based on the personalization factor.

In some embodiments generating the at least one personalized abnormality criterion comprises: calculating a personalized variability measure based on the general variability measure and the personalization factor; and defining at least one personalized abnormality criterion based on the personalized variability measure.

In some embodiments the method further comprises receiving at least one further measured value having an earlier measurement time than the received measured value. In some such embodiments determining whether the received measured value is abnormal comprises assessing whether the at least one further measured value meets the at least one abnormality criterion. In some such embodiments determining the received measured value to be abnormal if both of the at least one further measured value and the received measured value meet the at least one abnormality criterion.

In some embodiments the general variability measure comprises the standard deviation of the historical data set.

In some embodiments wherein the personalization factor comprises a scaling factor.

In some embodiments the physiological data relating to the subject comprises one or more of the following data types: a determination of the right ventricular ejection fraction, RVEF, and/or the left ventricular ejection fraction, LVEF, of the subject; a determined fitness level of the subject; health conditions experienced by the subject; a historical data set of measured values of the physiological characteristic obtained from the subject.

In some embodiments calculating the personalization factor comprises: comparing the physiological data relating to the subject to a data base correlating physiological data of the same type as the subject specific physiological data with variability of the physiological characteristic, to determine a subject-specific variability associated with the physiological data relating to the subject; and comparing the subject-specific variability with the general variability measure.

In some embodiments the physiological characteristic is bio-impedance.

There is also provided, according to a second aspect of the invention, a computer program product, comprising computer readable code embodied therein, the computer readable code being configured such that, on execution by a suitable computer or processor, the computer or processor performs the method according to the first aspect.

There is also provided, according to a third aspect of the invention, an apparatus for use in monitoring a physiological characteristic of a subject, the apparatus comprising:

a processing unit arranged to:

obtain a general variability measure of the physiological characteristic, wherein the general variability measure is based on a historical data set of values of the physiological characteristic from a plurality of further subjects;

calculate a personalization factor specific to the first subject, based on physiological data relating to the first subject;

generate at least one personalized abnormality criterion for the physiological characteristic, based on the obtained general variability measure and the calculated personalization factor, wherein a given received measured value is determined to be abnormal if it meets the at least one personalized abnormality criterion;

receive a measured value of the physiological characteristic of the subject; and

determine whether the received measured value is abnormal by comparing it to the at least one personalized abnormality criterion.

There is also provided, according to a fourth aspect of the invention, a system for use in monitoring a physiological characteristic of a subject, the system comprising: a measurement device for measuring a physiological characteristic of a subject, wherein the measurement device is arranged to output a measured value of the physiological characteristic; and an apparatus according to the third aspect, wherein the processing unit is arranged to receive a measured value output by the measurement device.

Various other embodiments of the apparatus are also contemplated in which the processing unit is further configured to execute any of the above-described method steps.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 is an illustration of an apparatus for measuring the fluid content of part of the body of a subject according to an embodiment;

FIG. 2 is a flow chart illustrating a method for use in monitoring a physiological characteristic of a subject according to a general embodiment of the invention; and

FIG. 3 is a flow chart illustrating the implementation of steps 103 and 105 of FIG. 2 according to a specific embodiment of the invention.

### DETAILED DESCRIPTION OF THE PREFERRED EMBODIMENTS

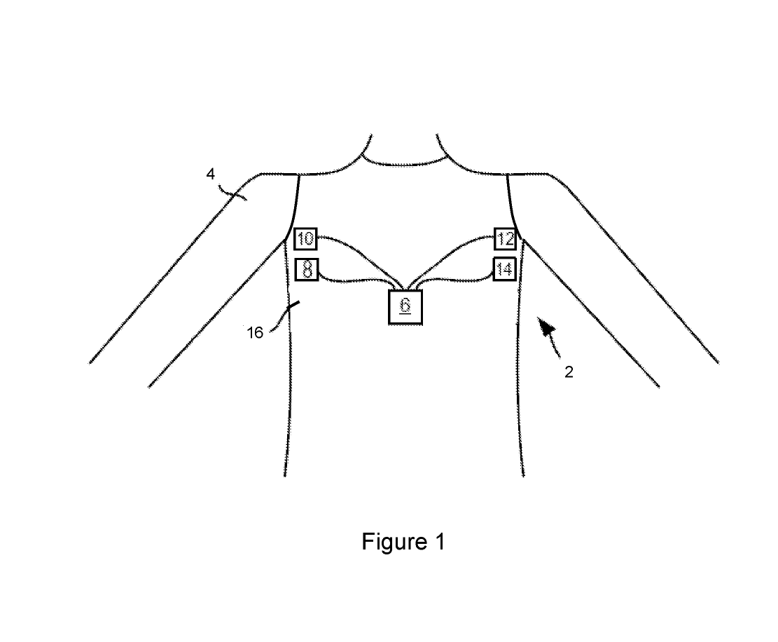

FIG. 1 shows an apparatus for use in monitoring a physiological characteristic of a subject according to the invention. In this particular embodiment, the physiological characteristic is the fluid content of a part of the body of a subject, measured in terms of the resistance of the part of the body of the subject, and thus FIG. 1 shows an measurement device for monitoring fluid content in a part of the body of a subject (patient) using bio-impedance measurements that can implement the method according to the invention. However, those skilled in the art will appreciate that the apparatus can be readily adapted for use in monitoring other physiological parameters through the inclusion or use of a suitable sensor. For example, the apparatus can be used to monitor any of a variety of other physiological characteristics, such as blood pressure, temperature, heart rate, heart rate variability, heart sounds, lung sounds, blood oxygen saturation (SpO₂) etc. using sensors suitable for measuring those characteristics.

The measurement device 2 is shown in FIG. 1 as being incorporated in a vest 16 worn by a subject 4, although it will be appreciated that other arrangements are possible, such as a strap or belt to which the measurement device is attached. The measurement device 2 comprises a control unit 6 that is connected to electrodes 8, 10, 12, 14 that are to be attached to, or otherwise placed into contact with, the skin of the subject 4. In the illustrated embodiment two pairs of electrodes are provided, although it is possible to use more or fewer electrode pairs. A first pair of electrodes 8, 12 deliver a small electric current at one or more selected frequencies into the chest of the subject 4. A second pair of electrodes 10, 14, each placed near a respective one of the electrodes 8, 12 in the first pair, are placed on the skin of the subject 4 to measure the differential potential across the chest of the subject 4. Where, as in the illustrated embodiment, the measurement device 2 is to monitor the fluid accumulation in the lungs of the subject 4, the electrodes in each pair are placed on opposite sides of the thorax of the subject 4. The illustrated measurement device 2 is therefore a non-invasive trans-thoracic bio-impedance measurement system.

If the tissue in the body part being measured contains a high level of fluid, it conducts electricity better and thus its impedance is lower than if it contains less fluid. By measuring the impedance at different frequencies the resistance of extracellular fluid (Rₑ) in the tissue can be estimated separately from the resistance of the intracellular fluid (Rᵢ) using the Cole-Cole model. In particular, at low measurement frequencies (e.g. approaching 0 Hz) the measured biological tissue impedance is mainly determined by the extracellular fluid content and its characteristics. At these low frequencies, the injected current does not easily pass through cell membranes. At higher frequencies the electrical properties of the biological tissue are determined by both the intracellular and extracellular fluid content as the injected current is able to pass through the cell membranes. Therefore, the influence of the intra- and extra-cellular fluid content on the measured bio-impedance depends on the frequency of the injected current. This allows a characterization of the electrical properties of the biological tissue according to the Cole-Cole model. Using measurements at multiple frequencies allows the approximation by interpolation of the electrical properties of the tissue at direct current (DC, frequency of zero Hz) when the extracellular fluid content is the main component of the impedance.

Where, in alternative or additional embodiments, the measurement device 2 is to be used to monitor the subject for dehydration, the overall resistance of the tissue (i.e. including both the intra-cellular Rᵢ and extra-cellular Rₑ components) or the intra-cellular resistance Rᵢ can be determined from the measured biological tissue impedance.

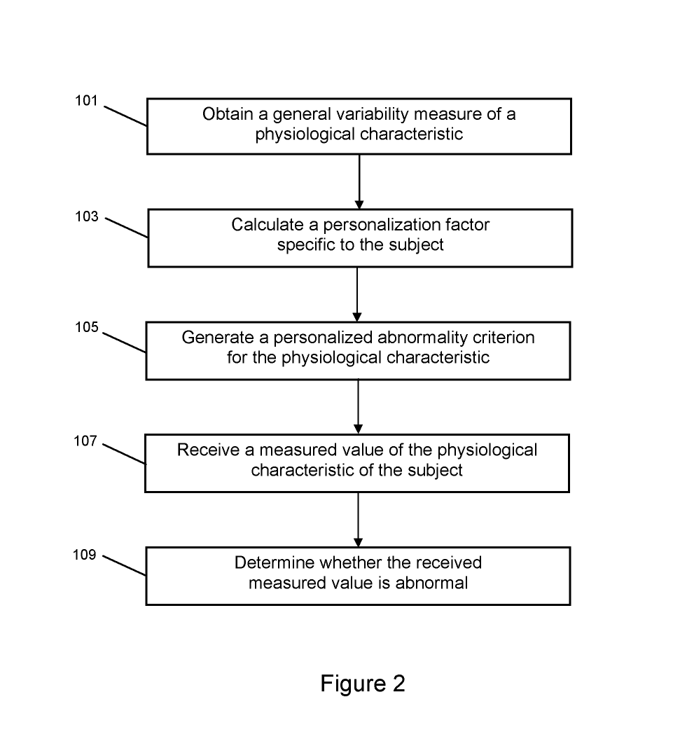

The estimation of Rₑ and/or Rᵢ values from impedance measurements can be performed by a processing unit in the control unit 6. The control unit 6 can also implement the method described below and shown in FIG. 2. Alternatively, the control unit 6 can transmit the impedance measurements or estimated Rₑ and/or Rᵢ values to another apparatus (such as a smart phone, laptop computer, desktop computer or other processing device) that comprises a processing unit that processes the impedance measurements to produce Rₑ and/or Rᵢ values (if required) and that implements the other steps in the method shown in FIG. 2.

In alternative implementations, an apparatus that comprises two spatially and electrically separated electrodes for surgical implantation into the tissue of a subject's body can be used to collect the bio-impedance measurements. The person skilled in the art will also be aware of other types of systems to that shown in FIG. 1 that can be used to provide bio-impedance measurements.

FIG. 2 shows a method for use in monitoring a physiological characteristic of a subject according to a general embodiment of the invention.

In step 101, a general variability measure of the physiological characteristic is obtained. The general variability measure is based on a historical data set of values of the physiological characteristic from a population of subjects, and therefore indicates the variability of the physical characteristic across that population of subjects. In preferred embodiments the population of subjects comprises subjects known to have a health condition associated with variations in the physiological characteristic, and the values in the data set comprise values measured when the subjects were in a stable state with respect to the health condition. For example, in embodiments where the physiological characteristic is bio-impedance, preferably the population of subjects comprises stable chronic heart failure patients. In preferred embodiments the general variability measure is a statistical measure of the variability of the historical data set, such as a variance, a standard deviation, or an interquartile range of the historical data set.

In a specific embodiment a processing unit of a monitoring apparatus calculates a general variability measure in the following manner. The historical data set includes historical Rₑ values for the population of subjects (this data set will hereafter be referred to as Stable_Rₑ). An algorithm executed by the processing unit computes several parameters from the Rₑ values in Stable_Rₑ. In particular, an average (e.g. the mean Rₑ), a measure of the variation (e.g. the standard deviation of Rₑ) and maximum and minimum coefficients of variance CVₘₐₓ and CVₘᵢₙ respectively are calculated (CVₘₐₓ and CVₘᵢₙ are calculated by estimating the confidence intervals at a confidence level (e.g. 50%, 95%, 99%, etc.) of the coefficient of variance of the Rₑ values). The general variability measure is then computed according to the following equation:

HV_{Gen}=std(Stable_Rₑ)

If HV_{Gen}<(CVₘᵢₙ*EHV) then HV_{Gen}=CVₘᵢₙ*EHV

If HV_{Gen}>(CVₘₐₓ*EHV) then HV_{Gen}=CVₘₐₓ*EHV (Equation 1)

where HV_{Gen} is the general variability measure, std(Stable_Rₑ) is the standard deviation of the set Stable_Rₑ, and EHV is the calculated average of the set Stable_Rₑ. It may, in certain embodiments, be desirable to calculate a range of normal variability of Rₑ (defined in terms of an upper bound UB and a lower bound LB) based on the general variability measure. In such embodiments the lower bound LB and upper bound UB of the range of normal variability can be calculated according to:

LB=EHV−HV_{Gen}

UB=EHV+HV_{Gen} (Equation 2)

It will be appreciated that the range output by Equation 2 is based only on the data set Stable_Rₑ, and is therefore not personalized to the subject.

In some embodiments obtaining the general variability measure comprises calculating the general variability measure, e.g. with a processing unit of the monitoring apparatus 2. In other embodiments obtaining the general variability measure comprises retrieving the general variability measure from a memory, e.g. of the measurement device 2 or of a central server located at a healthcare provider.

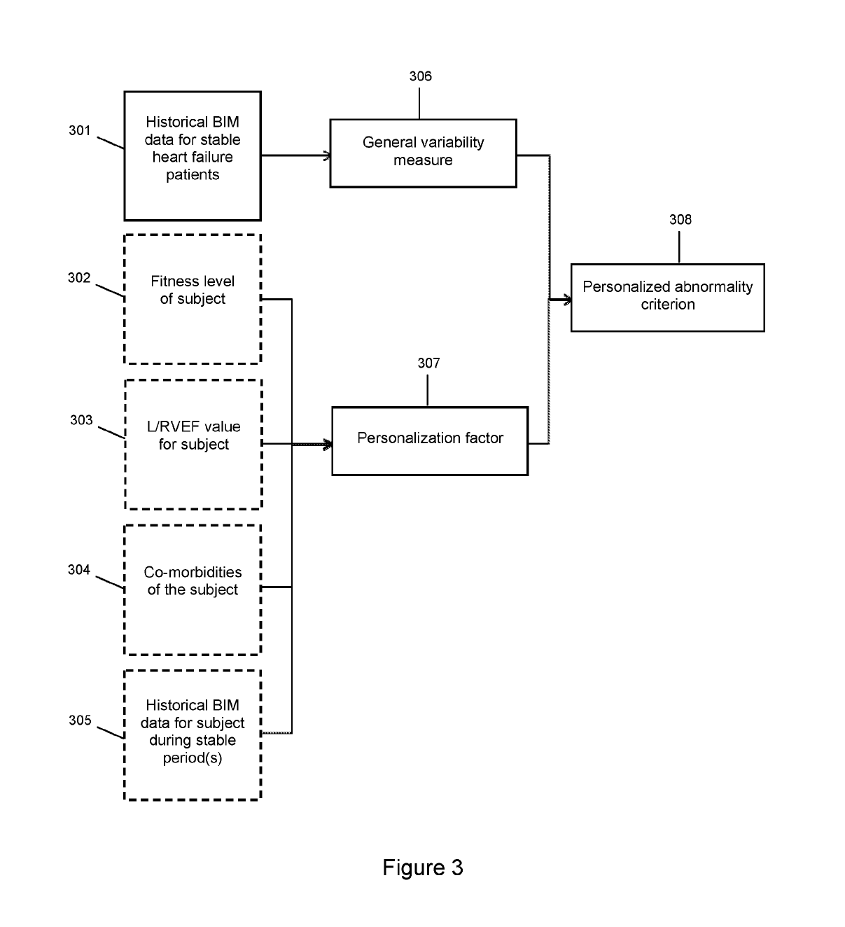

In step 103, a personalization factor specific to the subject is calculated, e.g. by the processing unit of the measurement device 2 or by a central processing unit at the healthcare provider. The personalization factor is calculated based on physiological data relating to the subject. Various different ways of calculating the personalization factor are envisaged. In preferred embodiments the physiological data comprises a single measurement and/or test result, or a set of measurements and/or test results obtained simultaneously or over a short time period (preferably less than a day). However; in other embodiments the physiological data comprises a time series of measurements and/or test results. In preferred embodiments the personalization factor comprises a scaling factor. Particular exemplary methods of calculating a personalization factor will be discussed in more detail below, in reference to FIG. 3.

The measurement(s) and/or test result(s) used in step 103 can be obtained either using the measurement device that is subsequently used to provide measurement data that is analyzed for abnormality, or a different measurement device to that used to subsequently monitor the subject for abnormality. Alternatively, the measurement(s) and/or test result(s) used in step 103 could be measured using multiple measurement devices that measure bio-impedance of the subject and/or other physiological parameters (e.g. one measurement device can be used while the subject is in hospital, and a different measurement device can be used while the subject is being monitored for abnormality at home). It will be appreciated that it is possible for the measurement(s) and/or test result(s) to be (or have been) obtained when the subject is not directly being monitored for abnormal measurements (e.g. when the subject is recovering from an abnormal status back to their normal range, such as when the subject is recovering in hospital).

In step 105, at least one personalized abnormality criterion for the physiological characteristic is generated. The at least one personalized abnormality criterion is generated based on the general variability measure and the personalization factor. A given measured value of the physical characteristic is determined to be abnormal if it meets the at least one personalized abnormality criterion. In some embodiments the criterion comprises a range of healthy values of the physiological characteristic, and is defined such that any value falling outside this range is determined to be abnormal. In some embodiments the criterion comprises a maximum threshold such that any value exceeding the maximum threshold is determined to be abnormal. In other embodiments the criterion comprises a minimum threshold such that any value falling below the minimum threshold is determined to be abnormal. It will be appreciated that the above-mention criteria are examples of value-based criteria, because in each case whether or not a given measured value meets the criterion depends on the value itself.

The definition of the at least one abnormality criterion will also depend on whether the method is being used to monitor for high values, low values, or both low and high values of the physiological characteristic. For example, some physiological conditions, such as excess fluid accumulation in the lungs, result in lower bio-impedance values due to excess extracellular water in the body of the subject. In this case, where the method is monitoring the bio-impedance of the subject to detect excess fluid accumulation (and also recovery from excess fluid accumulation), the abnormality criterion will be directed to generally treat low values as being abnormal. However, other physiological conditions, such as dehydration, result in higher bio-impedance values. Therefore if the method is used to monitor for dehydration, the abnormality criterion will be directed to generally treat high bio-impedance values as being abnormal.

In some embodiments the at least one abnormality criterion comprises a set of criteria, which must all be met by a given measured value in order for that value to be determined as abnormal. In some such embodiments the set of criteria comprises a time-based criterion and a value-based criterion. A time-based criterion may, for example, specify a minimum amount of time for which the measured values must have consistently met a value-based criterion in order for the most-recently acquired measured value to be determined as abnormal. Alternatively, a time-based criterion can specify a minimum number of consecutive measured values which must have met a value-based criterion in order for the most recent of the consecutive measured values to be determined as abnormal. Providing a time-based criterion can be advantageous because in some embodiments, a number of consecutive high or low values (as appropriate for the physiological characteristic and/or health condition being monitored) may need to be received before an accurate determination can be made that a high or low received measured value is abnormal. As a consequence of not immediately determining a received measured value which meets a value-based abnormality criterion to be abnormal, the number of false alarms caused by erroneous measurements (e.g. due to incorrect sensor placement or inconsistent measurement conditions) is reduced.

In determination of the abnormality criterion, for example as described below, it is possible to utilize multiple measurements and/or test results measured from the subject as described earlier. In particular, it can be useful in some embodiments to use measurements to determine the abnormality criterion that have been measured when the subject is not under surveillance (i.e. not being monitored) for abnormal measurement values but rather recovering back to the normal range.

In some embodiments the at least one personalized abnormality criterion is generated by defining at least one general abnormality criterion (such as the range of normal variability of Rₑ which is output by equation 2) and then adjusting the at least one general abnormality criterion based on the personalization factor. For example, in the above discussed first specific embodiment (i.e. the embodiment of equations 1 and 2), a “general” range of healthy values of the physiological characteristic is defined based on the general measure of variability. It will be appreciated that the general range can be personalized by altering the width and/or mid-point value of the range. In some embodiments the personalization factor is used to alter the width of the range without altering its mid-point (it can be seen from equation 2 that this can be achieved by applying a scaling factor to HV_{Gen}). Alternatively, the mid-point of the range can be altered without changing the width by applying a scaling factor to EHV. In some embodiments a personalization factor is applied such that both the width and the mid-point are altered (it will be appreciated that in embodiments where a scaling factor is applied to both HV_{Gen} and EHV, a different scaling factor may be used for each). In some embodiments the general abnormality criterion comprises a threshold, in which case the personalization factor is used to adjust the value of the threshold.

It will be appreciated that the value of the scaling factor will depend on whether the physiological data on which the personalization factor is based indicates that the subject is likely to exhibit more or less variation in the physiological characteristic than is typical. Applying the scaling factor to the general range therefore produces a personalized range of healthy values, which is specific to the subject, and can therefore be used to generate one or more personalized abnormality criteria. As with the abnormality criterion, the scaling factor can be determined, for example, using measurements from the subject obtained either during a normal surveillance (monitoring) period when the subject is moving towards abnormal or when the subject is recovering from an abnormal state and moving towards normal state (e.g. when the subject is in hospital).

In other embodiments the at least one personalized abnormality criterion is generated by applying the personalization factor directly to the general variability measure to produce a personalized variability measure, and then generating at least one personalized abnormality criterion based on the personalized variability measure. For example, in some such embodiments the personalization factor comprises a scaling factor, and a statistical measure of the variability of the historical data set is scaled by this scaling factor before the abnormality criterion is generated. It will be appreciated that in some situations (e.g. where the abnormality criterion comprises a range of healthy values based on the interquartile range of the historical data set), the resulting personalized abnormality criterion will be the same regardless of whether the personalization factor is applied directly to the general variability measure or is applied to a general abnormality criterion based on the general variability measure, but that this will not necessarily always be the case.

In step 107 a new measured value of the physiological characteristic of the body of the subject is received or obtained, for example from bio-impedance measurement device 2 which measures the impedance of tissue with a small alternating current (AC) flowing between two electrodes. The voltage drop between the electrodes can be measured either between two other electrodes (four-point measurement) or with the same ones as are used to inject the current. In some embodiments, the value of the physiological characteristic is the value of the resistance of fluid in a part of the subject's body. In preferred embodiments, the value of the resistance of fluid is the resistance Rₑ of extracellular fluid in the part of the body of the subject, and more preferably, Rₑ is the resistance of extracellular fluid in the lungs of the subject. However, in alternative embodiments, the value of the physiological characteristic can be a weight measurement, heart rate measurement, blood pressure measurement or temperature measurement, etc.

In step 109, the received measured physiological characteristic value is compared to the at least one personalized abnormality criterion and it is thereby determined whether the received measured value is abnormal. In preferred embodiments, if the received measured value meets the at least one abnormality criterion it is determined to be abnormal. In some embodiments if the received measured value does not meet the at least one abnormality criterion, or does not meet all of a set of abnormality criteria, the received measured value is determined to be normal.

In some embodiments, if the received measured physiological characteristic value is determined to be abnormal, then the measurement can be flagged as such (e.g. reported to the subject or physician). In some embodiments, particularly where the method is directed to identify excess extracellular fluid accumulation, an indication of a physiological condition which is correlated to the physiological characteristic (in this case the fluid content or accumulation in the part of the subject's body) can be determined from the physiological characteristic value.

FIG. 3 illustrates an exemplary process for generating the at least one personalized abnormality criterion, according to a specific embodiment of the invention. In the specific embodiment, the physiological characteristic being measured is bio-impedance, which can indicate heart failure exacerbations as discussed previously. A general bio-impedance variability measure 306 is obtained based on a database 301 of historical bio-impedance values from a population of stable heart failure patients. In this embodiment the general variability measure is equal to the standard deviation of the bio-impedance values in the database 301.

A personalization factor 307 is calculated, based on physiological data specific to the subject. The type of the physiological data used in the calculation of the personalization factor is selected based on which health condition is of interest. In the FIG. 3 embodiment the health condition of interest is heart failure (although it will be appreciated that bio-impedance measurements can also be informative about other health conditions, such as dehydration). As such, the personalization factor 307 is calculated based on physiological data known to be affected by heart failure.

Experimental evidence exists showing how fitness tests can be used to determine cardio-vascular fitness, and cardio-vascular fitness is known to be related to the state of heart failure (see, e.g., Tanino, Y. et al., “Whole Body Bioimpedance Monitoring for Outpatient Chronic Heart Failure Follow-up”, Circulation: 73, 2009). This paper also shows that if bio-impedance is used for cardiac output estimation, the estimate has a statistically significant (although weak) correlation with the ventricular ejection fraction (VEF). This suggests that the left ventricular ejection fraction (LVEF) or right ventricular ejection fraction (RVEF) of a subject can be used to estimate their normal bio-impedance variation range. Advantageously, a fitness test result and/or an L/RVEF value for a given subject can be obtained immediately, without requiring a sustained period of monitoring.

A personalization factor can be generated based on a fitness test result 302 by comparing the fitness of the subject to a data base correlating fitness with bio-impedance variability (for healthy and/or stable subjects). It can then be ascertained whether the bio-impedance variability corresponding to the subject's fitness level is greater or less than the general variability measure 306 and the personalization factor 307 (which in this embodiment is a scaling factor) can be defined accordingly. In one embodiment, for example, the personalization factor 307 is calculated according to the equation:

P=HV_{fit}/HV_{Gen} (Equation 3)

where P is the personalization factor, HV_{fit} is the bio-impedance variability corresponding to the subject's fitness level 302, and HV_{Gen} is the general variability measure.

Alternatively, the fitness level 302 of the subject can be used to estimate a scaling factor in an adhoc manner, based on the assumption that a low bio-impedance value in a subject with a higher fitness level is less likely to indicate a heart failure exacerbation. The personalization factor 307 can thereby be defined such that it increases the general variability measure if the subject is fitter than a predefined baseline fitness, or decreases the general variability measure if the subject is less fit than the predefined baseline. In such embodiments the value of the personalization factor 307 (and therfore the amount of the increase/decrease) will depend on by how much the subject's fitness differs from the baseline.

In some embodiments a plurality of fitness categories are defined, each of which is associated with its own characteristic bio-impedance variation range. In some such embodiments the characteristic bio-impedance variation range for each fitness category is calculated as a percentage above and below the baseline and the personalization factor 307 is calculated as:

P=HV_{fit}Rₑ (Equation 4)

The same methods can be used to calculate a personalization factor 307 using a measured L/RVEF 303 of the subject.

The state of heart failure is also expected to be related to co-morbidities (i.e other health conditions simultaneously experienced by a heart failure patient). The provision of a data set relating co-morbidity data to a measured physiological characteristic would therefore enable a personalization factor 307 to be calculated based on the co-morbidities 304 a subject is known to be experiencing. For example, if diabetes can be shown to be linked to increased variability in measured bio-impedance, then a personalization factor calculated for a diabetic subject would increase the general variability measure (assuming the calculation did not take into account any other patient specific physiological data). Existing co-morbidities of a monitored subject will typically be known from the outset of the monitoring, so a personalization factor can be created immediately on this basis. To assess the impact of the co-morbidities, a database collected of known values of bio-impedance variability categorised by co-morbidities can be used in the same manner as Equation 4 suggests for fitness tests.

Existing historical data 305 from the subject can also be used in the calculation of the personalization factor 307, if it is available. Historical bio-impedance values acquired during periods when the subject was stable (in relation to their heart failure) can be used to directly determine a personalized bio-impedance variability measure for the subject, using the same techniques as used to determine the general variability measure 306. A personalization factor 307 can then be calculated by comparing the personalized variability measure to the general variability measure, e.g. using the equation:

P=HV_{Per}/HV_{Gen} (Equation 5)

where HV_{Per} is the personalized variability measure.

In some embodiments, the historical data 305 may also include or indicate the response of the subject to the measured bio-impedance values in the normal state of the subject, in the abnormal state of the subject, in transitions from normal to abnormal (e.g. deterioration) and/or also in transitions from abnormal back to normal (e.g. recovery). The response data can indicate, e.g. the speed or rate at which the transition occurred and/or the length of time over which the deterioration and/or recovery occurred. This response data can be used in the calculation of the personalization factor 307.

Several types of physiological data can be used simultaneously in the calculation of the personalization factor, as is shown by FIG. 3. In this embodiment a fitness test result 302, an VEF value 303, co-morbidity information 304 and historical bio-impedance data 305 from stable (and/or recovery) periods are all available for the subject, and are all taken into account in the calculation of the personalization factor. In this embodiment an individual scaling factor is calculated for each of the different types of subject-specific physiological data, and these scaling factors are combined to produce an overall personalization factor 307. In some embodiments the overall personalization factor is an average of the individual scaling factors. In preferred such embodiments the overall personalization factor is a weighted average of the individual scaling factors, where different weights are assigned to the individual scaling factors in dependence on, e.g., the strength of the correlation between the physiological data type and heart failure state. It will be appreciated that, alternatively, the calculation of the personalization factor 307 could be based on any of the individual subject-specific physiological data types 302-303 in isolation, or on any possible combination thereof. It will also be appreciated that any other type of physiological data which correlates with heart failure state could be used instead or additionally in the calculation of the personalization factor 307. In this context, as noted above, both measurement data collected during a normal to abnormal transition (deterioration) or abnormal to normal transition (recovery), could be relevant and beneficial in determining the personalization factor 307.

Once the personalization factor 307 has been calculated, a personalized abnormality criterion 308 can then be calculated based on both the general variability measure 306 and the personalization factor 307. In the specific embodiment illustrated by FIG. 3, the personalized abnormality criterion comprises a minimum threshold for bio-impedance, such that bio-impedance values falling below this threshold are determined to be abnormal. The level of the minimum threshold is calculated by determining a baseline bio-impedance value (which may be subject-specific, but need not be). In the FIG. 3 embodiment, the baseline bio-impedance value comprises the mean of the historical subject-specific stable bio-impedance data 305 and is therefore personalized to the subject. In alternative embodiments (e.g. where subject-specific historical bio-impedance data is not available) the baseline bio-impedance value comprises the mean of the historical data set used to calculate the general variability measure 306. The personalization factor 307 is then used to scale the general variability measure 306, and the scaled general variability measure is subtracted from the baseline bio-impedance value to generate a personalized minimum threshold.

There is therefore provided a method and apparatus that allow a subject to be monitored so as to detect and/or predict the onset of or recovery from clinically significant conditions using measurements of one or more physiological characteristics of the subject.

While the invention has been illustrated and described in detail in the drawings and foregoing description, such illustration and description are to be considered illustrative or exemplary and not restrictive; the invention is not limited to the disclosed embodiments.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A method for use in monitoring a physiological characteristic of a subject, the method comprising:
obtaining a general variability measure of the physiological characteristic, wherein the general variability measure is based on a historical data set of values of the physiological characteristic from a plurality of further subjects;
calculating a personalization factor specific to the subject, based on physiological data relating to the subject, wherein the physiological data relating to the subject comprises one or more of a determination of the right ventricular ejection fraction, RVEF, and/or the left ventricular ejection fraction, LVEF, the subject; a determined fitness level of the subject; and health conditions experienced by the subject;
generating at least one personalized abnormality criterion for the physiological characteristic based on the obtained general variability measure and the calculated personalization factor;
receiving a measured value of the physiological characteristic of the subject; and
determining whether the received measured value is abnormal by comparing it to the at least one personalized abnormality criterion, wherein the received measured value is determined to be abnormal if it meets the at least one personalized abnormality criterion.

2. A method as claimed in claim 1, wherein the at least one personalized abnormality criterion comprises one or more of:
a personalized maximum value of the physiological characteristic, such that a received measured value which is above the maximum value is determined to be abnormal; and
a personalized minimum value of the physiological characteristic, such that a received measured value which is below the minimum value is determined to be abnormal.

3. A method as claimed in claim 1, wherein the at least one personalized abnormality criterion comprises a personalized range of normal values of the physiological characteristic, such that a received measured value which is outside the range of normal values is determined to be abnormal.

4. A method as claimed in claim 1, wherein generating the at least one personalized abnormality criterion comprises determining a baseline value of the physiological characteristic, and wherein the at least one personalized abnormality criterion is generated based on the determined base line value, the obtained general variability measure, and the calculated personalization factor.

5. A method as claimed in claim 4, wherein the baseline value is a personalized baseline value, which is determined using data specific to the subject.

6. A method as claimed in any preceding claim, wherein generating the at least one personalized abnormality criterion comprises:
defining at least one general abnormality criterion based on the obtained general variability measure; and
adjusting the at least one general abnormality criterion based on the personalization factor.

7. A method as claimed in claim 1, wherein generating the at least one personalized abnormality criterion comprises:
calculating a personalized variability measure based on the general variability measure and the personalization factor; and
defining at least one personalized abnormality criterion based on the personalized variability measure.

8. A method as claimed in claim 1, comprising receiving at least one further measured value having an earlier measurement time than the received measured value, and wherein determining whether the received measured value is abnormal comprises assessing whether the at least one further measured value meets the at least one abnormality criterion, and determining the received measured value to be abnormal if both of the at least one further measured value and the received measured value meet the at least one abnormality criterion.

9. A method as claimed in claim 1, wherein the general variability measure comprises the standard deviation of the historical data set.

10. A method as claimed in claim 1, wherein the personalization factor comprises a scaling factor.

11. (canceled)

12. A method as claimed in claim 1, wherein calculating the personalization factor comprises:
comparing the physiological data relating to the subject to a data base correlating physiological data of the same type as the physiological data relating to the subject with variability of the physiological characteristic, to determine a subject-specific variability associated with the physiological data relating to the subject; and
comparing the subject-specific variability with the general variability measure.

13. A computer program product, comprising computer readable code embodied therein, the computer readable code being configured such that, on execution by a suitable computer or processor, the computer or processor performs the method described in claim 1..

14. An apparatus for use in monitoring a physiological characteristic of a subject, the apparatus comprising:
a processing unit arranged to:
obtain a general variability measure of the physiological characteristic, wherein the general variability measure is based on a historical data set of values of the physiological characteristic from a plurality of further subjects;
calculate a personalization factor specific to the first subject, based on physiological data relating to the first subject, wherein the physiological data relating to the subject comprises one or more of a determination of the right ventricular ejection fraction, RVEF, and/or the left ventricular ejection fraction, LVEF, of the subject; a determined fitness level of the subject; and health conditions experienced by the subject;
generate at least one personalized abnormality criterion for the physiological characteristic, based on the obtained general variability measure and the calculated personalization factor, wherein a given received measured value is determined to be abnormal if it meets the at least one personalized abnormality criterion;
receive a measured value of the physiological characteristic of the subject; and
determine whether the received measured value is abnormal by comparing it to the at least one personalized abnormality criterion.

15. A system for use in monitoring a physiological characteristic of a subject, the system comprising:
a measurement device for measuring a physiological characteristic of a subject, wherein the measurement device is arranged to output a measured value of the physiological characteristic; and
an apparatus according to claim 13, wherein the processing unit is arranged to receive a measured value output by the measurement device.
